(19) United States
(12) Patent Application Publication
(10) Pub. No.: US 2006/0035913 A1
(43) Pub. Date: Feb. 16, 2006

(54) Method for the production of 2-amino-4-chloro-6-alkoxypyrimidines

(76) Inventors: Sylvia Huber, Trostberg (DE); Thomas Guthner, Trostberg (DE); Wolfgang Moser, Trostberg (DE); Doris Krammer, Truchtlaching (DE)

Correspondence Address: FULBRIGHT & JAWORSKI, LLP, 666 FIFTH AVE, NEW YORK, NY, 10103-3198, US

(21) Appl. No.: 10/528,959

(22) PCT Filed: Oct. 24, 2003
(86) PCT No.: PCT/EP03/11844

§ 371 Date: Mar. 22, 2005

(30) Foreign Application Priority Data
Oct. 26, 2002 (DE) 102 49 946.2

(51) Int. Cl. A61K 31/513; C07D 239/47

(52) U.S. Cl. 514/269; 544/320

(57) ABSTRACT

The present invention provides a process for preparing 2-amino-4-chloro-6-alkoxypyrimidines by reacting the 2-amino-4,6-dichloropyrimidine with an alkali metal alkoxide or a mixture of alkali metal hydroxides and an alcohol, in which the reaction is effected in a polar aprotic solvent (or solvent mixture), the solvent is subsequently distilled off to an extent of >30% and the product is precipitated by adding water during or after the distillation. This process, in which the polar aprotic solvent used may in particular be acetone and which can be performed at temperatures between 5 and 60° C., allows 2-amino-4-chloro-6-alkoxypyrimidines and in particular 2-amino-4-chloro-6-methoxypyrimidine to be prepared in high yields and at simultaneously very marked purity in a particularly economically viable and environmentally benign manner.

The present invention provides a process for preparing 2-amino-4-chloro-6-alkoxypyrimidines.

2-Amino-4-chloro-6-methoxypyrimidine (ACMP) is an important intermediate in the synthesis of highly active herbicides and various active pharmaceutical ingredients. Usually, ACMP is reacted with sulfonyl isocyanates or phenyl sulfonylcarbamates and is therefore also found in numerous sulfonylurea herbicide derivatives (e.g. DE-C 43 04 864, EP-A 246 984, BR 8303322, CN 1277195, CN 1171197, DE-A 197 07 580, U.S. Pat. No. 5,723,409, DE-C 43 41 454, EP-A 464 838, U.S. Pat. No. 4,699,647, EP-B 238 070, EP-B 232 067, U.S. Pat. No. 4,656,273, EP-A 156 521, EP-A 161 905, DE-A 31 51 450). ACMP is also used as an intermediate in active pharmaceutical ingredients; for example in diabetes medicaments (cf. WO 01/36 416) or anticancer medicaments (Zhenghou Daxue, Ziran Kexueban (2000), 32 (2), 87-88). The use of 2-amino-4-chloro-6-ethoxypyrimidine is described, for example, in connection with the synthesis of herbicides (EP-A 101 308, JP 62111982), Huaxue Shiji (1999), 21 (2), 73-75), and 2-amino-4-chloro-6-n-propoxypyrimi is used, for example, as an intermediate in the synthesis of active pharmaceutical ingredients (J. Chem. Med. (1986) 19 (5) 676-81).

The literature has already disclosed some processes for preparing 2-amino-4-chloro-6-methoxypyrimidine. Typically, the industrially available 2-amino-4,6-dichloropyrimidine (ADCP) is reacted with sodium methoxide (Rose et al., J. Chem. Soc, 1946, 81, 84), or methanol and sodium hydroxide or potassium hydroxide, or else potassium carbonate in methanol as a solvent (Kitani et al., Nippon Kagaku Zasshi, 74, 1953, 624). However, between 4 and 6% by weight of the reactant regularly remain in the product in the known reaction methods of ADCP in methanol or other solvents with methoxide, or with sodium hydroxide or potassium hydroxide and methanol, and first have to be removed by costly and inconvenient purification steps such as recrystallization or distillation and thus lead to a reduction in the yields. Thus, only yields of approximately 70% are achieved with the known processes.

The reaction of 2-amino-4,6-dichloropyrimidine with sodium ethoxide (J. Chem. Soc., 1946, 81, 84) or sodium propoxide (J. Med. Chem. 29, 5, 1986, 676-681) and the reaction with potassium hydroxide and ethanol (Nippon Kagaku Zasshi, 74, 1953, 624; Chem. Abstr. 1954, 13963) to give the corresponding 2-amino-4-chloro-6-ethoxy-pyrimidine and 2-amino-4-chloro-6-propoxypyrimidine respectively proceeds in a similar manner. However, satisfactory yields are not possible with this process either.

ACMP can also be prepared starting from N-cyano-cyanoacetimido methyl ester by reacting with a hydrogen halide (cf. JP 01016770). However, the yield in this process is only 60%.

The reaction of 2,4-dichloro-6-methoxypyrimidine with ammonia is also known (Gabriel et al., Chem. Ber., 36, 1903, 3383). However, one problem in this case is the lack of selectivity of the reaction and another is the inadequate availability of the raw material.

It has been found that 2-amino-4-chloro-6-alkoxypyrimidines and in particular ACMP cannot be prepared in the purity required under economically viable conditions by the processes known hitherto. It is therefore an object of the present invention to provide a process by which 2-amino-4-chloro-6-alkoxypyrimidines can be prepared by reacting with alkali metal alkoxide or a mixture of alkali metal hydroxides and an alcohol, without troublesome amounts of 2-amino-4,6-dichloropyrimidine reactant remaining in the product.

This object is achieved by the process according to the invention, in which the reaction is effected in a polar aprotic solvent or solvent mixture, the solvent or solvent mixture is subsequently distilled off to an extent of >30% and the product is precipitated during or after distillation by adding water.

It has been found in the process according to the invention, surprisingly, that 2-amino-4-chloro-6-alkoxypyrimidines can be obtained in an economically viable and environmentally benign manner from 2-amino-4,6-dichloropyrimidine in a purity of >98% and with ADCP contents of <0.2%, and the desired products are regularly obtained in yields of >95%.

It has been found to be advantageous to use a C₁-C₄-alcohol and most preferably methanol as the obligatory alcohol component, as a result of which especially the known ACMP is obtained.

According to the invention, the ADCP and the appropriate alkali metal alkoxide are used in a preferred molar ratio of 1:1 to 1.5 and more preferably of 1:1.05 to 1.10.

The process according to the invention is carried out in a polar aprotic solvent (or solvent mixture), but it is not restricted in any way to specific solvents within this scope. From the group of the polar aprotic solvents, particularly suitable solvents have been found to be those which are selected from the group of the ketones, amides or nitrites, and particular preference is given to using acetone, methyl ethyl ketone, dimethylimidazolidinone, cyclohexanone, dimethylformamide, N-methylpyrrolidone, acetonitrile and/or mixtures thereof.

Owing to its known low toxicity and the simple workup of the resulting mother liquor, acetone may be regarded as a particularly preferred solvent.

The reaction at temperatures between 5 and 60° C. and more preferably between 15 and 40° C. results in a particularly good selectivity of the process according to the invention.

The selectivity of the reaction necessary for the product is achieved in particular by reacting at low temperatures below about 20° C. and a limitation of the alkoxide or of the alkali metal hydroxide/alcohol mixture. 2-Amino-4,6-dichloropyrimidine is generally initially charged in the solvent and the alkoxide, for example methoxide, or the alkali metal hydroxide and the alcohol, for example methanol, is subsequently metered in.

For the subsequent reaction phase, the invention provides for the heating of the mixture to a higher temperature after the reactants have been added, more preferably to temperatures between 20 and 60° C. and in particular to temperatures between 25 and 45° C. This allows the reaction, if necessary, to be completed after addition and postreaction time have ended.

Subsequently, the solvent is distilled off to an extent of >30% in the process according to the invention, and it is to be regarded as preferred to distill off the solvent to an extent of more than 50% and more preferably to an extent of about 75 to 95%. This distillation step not only predominantly removes the solvent, but generally also advantageously removes excess alcohol, which additionally improves the yields.

The distillate may be recycled without any problem, as a result of which the amount of waste obtained in the process according to the invention is advantageously extremely low.

Subsequently, the product according to the present invention is precipitated by adding water. The addition of water may be carried out in the form of several portions as early as during the distillation, or after the distillation step, which is likewise provided for by the invention.

According to the invention, the procedure of addition in portions during the distillation is preferred, since it is possible in this way to distill off more solvent and higher yields can thus be achieved.

The salt formed in the reaction may either be removed, for example by filtration before the addition of water out of the polar aprotic solvent (acetone), and/or the salt may be dissolved in the mother liquor as a result of the addition of water, which is preferred in accordance with the invention. The product itself is typically isolated by filtration and, after the washing with water, subsequently dried under reduced pressure.

Additionally, it is possible in accordance with the invention, depending upon the quality of the 2-amino-4,6-dichloropyrimidine raw material used, to carry out a purification step with activated carbon. The addition is effected after the reaction and preferably before and/or during the distillation, and preference is given to stirring additionally for about one further hour under the conditions of the postreaction, i.e., for example, at temperatures between 20 and 60° C. In this case, the activated carbon is subsequently also filtered off together with the salt before the distillation, as a result of which all impurities are advantageously removed nearly fully from the raw material, in particular colored compounds or other troublesome by-products, for example 2-amino-4-methoxy-6-(4′,6′-dimethoxypyrimidin-2′-ylamino)pyrimidine. This activated carbon purification step allows a purely white product to be obtained, which additionally demonstrates its quality.

It is possible with the process according to the invention to obtain 2-amino-4-chloro-6-alkoxypyrimidines and in particular ACMP in a particularly economically viable and environmentally benign manner, in high yields and at simultaneously very marked purity.

The present invention provides a process for preparing 2-amino-4-chloro-6-alkoxypyrimidines by reacting the 2-amino-4,6-dichloropyrimidine with an alkali metal alkoxide or a mixture of alkali metal hydroxides and an alcohol, in which the reaction is effected in a polar aprotic solvent (or solvent mixture), the solvent is subsequently distilled off to an extent of >30% and the product is precipitated during or after distillation by adding water. It is possible with this process, in which the polar aprotic solvent used is in particular acetone and which can be performed at temperatures between 5 and 60° C., to obtain 2-amino-4-chloro-6-alkoxypyrimidines and in particular 2-amino-4-chloro-6-methoxypyrimidine in a particularly economically viable and environmentally benign manner, in high yields and simultaneously very marked purity.

The examples which follow demonstrate these advantages of the process according to the invention.

### EXAMPLES

### Example 1

### Inventive

1000 ml of acetone were initially charged in a reaction vessel and then 32.8 g of 97.6% ADCP (195 mmol) were introduced. The temperature of the suspension was adjusted to 17° C. and then 37.44 g of 30% sodium methoxide solution (0.208 mol) were added dropwise at 17° C. within approx. 3.5 hours. The mixture was stirred at this temperature for a further 1 hour and then heated to 30° C. for 2 hours. 0.8 g of activated carbon was added to the still-warm suspension which was stirred for approx. 1 hour. Subsequently, the activated carbon and the sodium chloride which had formed were filtered off. 700 ml of acetone were distilled off from the filtrate under reduced pressure and the distillation residue was subsequently admixed with 500 ml of water. After cooling to 7° C., the product was filtered off and washed once with 50 ml of water. After drying under reduced pressure at 50° C., 29.67 g of ACMP remained (yield 95.3%, purity 99.3% in comparison to external standard by HPLC; residual ADCP content <0.2% by weight).

### Example 2

### Inventive

1000 ml of acetone were initially charged in a reaction vessel and then 32.8 g of 97.6% ADCP (195 mmol) were introduced. The temperature of the suspension was adjusted to 17° C. and then 37.44 g of 30% sodium methoxide solution (0.208 mol) were added dropwise at 17° C. within 3.5 hours. The yellow suspension was stirred at this temperature for another 1 hour and then heated to 30° C. for two hours. Subsequently, 500 ml of acetone were distilled off from the suspension at up to 43° C. under reduced pressure; during the distillation, 600 ml of water were added in portions and the mixture was distilled further. After cooling to 7° C., the product was filtered off and washed twice with 50 ml of water. After drying, 30.09 g of ACMP remained (yield 96.7%, purity 99.8% in comparison to an external standard by HPLC; residual ADCP content <0.2% by weight).

### Example 3

### Inventive

250 ml of dimethyl formate were initially charged in a reaction vessel and then 41 g of 97.6% ADCP (244 mmol) were introduced. The temperature of the suspension was adjusted to 17° C. and then 46.8 g of 30% sodium methoxide solution (0.260 mol) were added dropwise at 17° C. within 3.5 hours. The suspension was stirred at this temperature for another 1 hour and then heated to 30° C. for 2 hours. 1 g of activated carbon was then added to the mixture which was stirred at 30° C. for a further 1 h. After the filtration, 500 ml of water were added to the filtrate which was cooled to 7° C. Subsequently, the product was filtered off and washed twice with 50 ml of water. After the drying, 36.84 g of ACMP remained (yield 94.6%, purity 97.10% in comparison to an external standard by HPLC; residual ADCP content <0.2% by weight).

### Example 4

### Comparative

500 ml of methanol were initially charged and 82 g of ADCP and potassium carbonate were introduced at room temperature. The mixture was heated and boiled under reflux for 2 h, as a result of which the reaction suspension became less viscous. After cooling, 370 ml of methanol were distilled off and 250 ml of water were added to the residue. After stirring at room temperature for 1 hour, the product was filtered off and washed three times with 75 ml each time of water. After drying under reduced pressure, 75.82 g of product remained (95% of theory, ACMP content 93.5% by HPLC in comparison to an external standard; residual ADCP content 4.5% by weight).

### Example 5

### Comparative

500 ml of methanol and 82 g of ADCP were initially charged and cooled to 17° C. 90 g (0.5 mmol) of sodium methoxide solution (30%) were then metered in within 3.5 h, and the mixture was subsequently heated and boiled under reflux for 1 h to complete the reaction. After cooling, 495 ml of methanol were distilled off and 250 ml of water were added to the residue. After stirring at room temperature for 1 hour, the product was filtered and washed three times with 75 ml each time of water. After drying under reduced pressure, 77.31 g of product remained (97% of theory; ACMP content 94.5% by HPLC in comparison to an external standard; residual ADCP content 4.2% by weight).

1. A process for preparing a 2-amino-4-chloro-6-alkoxy-pyrimidine by comprising reacting a 2-amino-4,6-dichloropyrimidine with an alkali metal alkoxide or a mixture of alkali metal hydroxides and an alcohol in a polar aprotic solvent or solvent mixture, wherein the solvent or solvent mixture is subsequently distilled off to an extent of >30% and the product is precipitated during or after distillation by adding water.

2. The process as claimed in claim 1, wherein the alcohol is a C₁-C₄-alcohol.

3. The process as claimed in claim 1, wherein the molar ratio of 2-amino-4,6-dichloropyrimidine and the alkali metal alkoxide is 1:1 to 1.5.

4. The process as claimed in claim 1, wherein the polar aprotic solvent is selected from the group consisting of a ketone, an amide, a nitrile and mixtures thereof.

5. The process as claimed in claim 1, wherein the reaction is effected at a temperature between 5 and 60° C.

6. The process as claimed in claim 1, wherein the mixture is heated to a higher temperature after the reactants have been added.

7. The process as claimed in claim 1, wherein the solvent is distilled off to an extent of more than 50%.

8. The process as claimed in claim 1, wherein activated carbon is added to the reaction mixture before, during or before and during the distillation.

9. The process as claimed in claim 1, wherein salts formed are removed or brought into solution by adding water, or both.

10. The process as claimed in claim 1, wherein the polar aprotic solvent is selected from the group consisting of acetone, methyl ethyl ketone, dimethylimidazolidinone, cyclohexanone, dimethyl-formamide, N-methylpyrrolidone, acetonitrile and mixtures thereof.

11. The process as claimed in claim 1, wherein the reaction is effected at a temperature between 15 and 40° C.

12. The process as claimed in claim 1, wherein the mixture is heated to a higher temperature of between 20 and 60° C. after the reactants have been added.

13. The process as claimed in claim 1, wherein the mixture is heated to a higher temperature of between 25 and 45° C. after the reactants have been added.

14. The process as claimed in claim 17, wherein the solvent is distilled off to an extent of from 75-95%.

15. The process as claimed in claim 2, wherein the polar aprotic solvent is selected from the group consisting of acetone, methyl ethyl ketone, dimethylimidazolidinone, cyclohexanone, dimethyl-formamide, N-methylpyrrolidone, acetonitrile and mixtures thereof.

16. The process as claimed in claim 3, wherein the polar aprotic solvent is selected from the group consisting of acetone, methyl ethyl ketone, dimethylimidazolidinone, cyclohexanone, dimethyl-formamide, N-methylpyrrolidone, acetonitrile and mixtures thereof

17. The process as claimed in claim 4, wherein the polar aprotic solvent is selected from the group consisting of acetone, methyl ethyl ketone, dimethylimidazolidinone, cyclohexanone, dimethyl-formamide, N-methylpyrrolidone, acetonitrile and mixtures thereof.

18. The process as claimed in claim 2, wherein alcohol is methanol.

19. The process as claimed in claim 3, wherein alcohol is methanol.

20. The process as claimed in claim 4, wherein alcohol is methanol.
